(19) United States
(12) Patent Application Publication
(10) Pub. No.: US 2017/0146510 A1
(43) Pub. Date: May 25, 2017

(54) DEVICES AND METHODS FOR ADJUSTABLE NANOGAP ELECTRODES

(71) Applicants: Quantum Biosystems Inc., Tokyo (JP); Osaka University, Osaka (JP)

(72) Inventors: Shuji Ikeda, Tokyo (JP); Masateru Taniguchi, Osaka (JP)

(21) Appl. No.: 15/344,184

(22) Filed: Nov. 4, 2016

Related U.S. Application Data
(63) Continuation of application No. PCT/JP2015/063964, filed on May 8, 2015.
(60) Provisional application No. 61/990,542, filed on May 8, 2014.
(60) Provisional application No. 61/990,507, filed on May 8, 2014.

(51) Int. Cl. G01N 33/487; G01N 27/447; C12Q 1/68

(52) U.S. Cl. CPC G01N 33/48721; C12Q 1/6869; G01N 27/44791

(57) ABSTRACT

The present disclosure provides methods and structures for effectuating nanoelectrodes with an adjustable nanogap. Devices with integrated actuators (e.g., piezoelectric devices) and/or materials with different coefficients of expansion are described. Also described are methods for calibrations nanoelectrode pairs.

### CROSS-REFERENCE

This application claims priority to U.S. Provisional Patent Application Ser. No. 61/990,542, filed May 8, 2014, and U.S. Provisional Patent Application Ser. No. 61/990,507, filed May 8, 2014, each of which is entirely incorporated herein by reference.

### DESCRIPTION OF THE RELATED ART

Nanopores may be useful for determining the sequence of a nucleic acid molecule, such as a deoxyribonucleic acid (DNA) or ribonucleic acid (RNA) molecule. The determination of the sequence of a nucleic acid molecule may provide various benefits, such as aiding in diagnosing and/or treating a subject. For example, the nucleic acid sequence of a subject may be used to identify, diagnose and potentially develop treatments for genetic diseases.

### SUMMARY OF THE INVENTION

As recognized herein, in some devices, multiple tunneling nanoelectrodes may be utilized in a single nanochannel so as to measure a nucleic acid molecule, such as a single-stranded nucleic molecule, several times, which may improve both the accuracy and speed of the measurements. In order to operate effectively, the nanoelectrode gaps, which may be of a few nanometers to less than a nanometer in width, and thus may need to be tightly controlled, potentially with tolerances of significantly less than a nanometer. In some cases, this may exceed the capabilities of current fabrication techniques, requiring the nanogap to be adjustable. It may be further desirable to adjust the gap width if a single device is utilized for several different types of biopolymers, wherein different gap widths may be needed for the different types biopolymers for the system to function optimally.

The present disclosure provides methods and apparatuses for creating adjustable nanoelectrode systems which may be used for sensing and/or sequencing a nucleic acid molecule, such as deoxyribonucleic acid (DNA) or ribonucleic acid (RNA), or sequencing and/or sensing other biopolymers, as well as detecting and identifying molecules. A nucleic acid sequencing device or system of the present disclosure can include tunneling nanoelectrodes, particularly adjustable tunneling nanoelectrodes, which may be used for determining the sequence of a nucleic acid molecule. The nucleic acid molecule can be single stranded or double stranded.

An aspect of the present disclosure provides a device for determining a sequence of a biopolymer, comprising: a substrate comprising at least one fluidic nanochannel; a plurality of electrode structures disposed adjacent to the substrate, each electrode structure of the plurality comprising at least one nanoelectrode pair, wherein each nanoelectrode pair comprises a region defining a gap between nanoelectrodes of the at least one nanoelectrode pair, and wherein the at least one nanoelectrode pair intersects the at least one fluidic nanochannel; and an actuator that is integrated with the at least one nanoelectrode pair, which actuator adjusts a spacing of the gap between the nanoelectrodes of the at least one nanoelectrode pair.

In some embodiments of aspects provided herein, the substrate is silicon. In some embodiments of aspects provided herein, the actuator is a piezoelectric element incorporated into the substrate. In some embodiments of aspects provided herein, the actuator is a piezoelectric element external to the substrate. In some embodiments of aspects provided herein, the gap is oriented at an angle substantially non-perpendicular to the substrate plane. In some embodiments of aspects provided herein, the actuator comprises a cantilever structure. In some embodiments of aspects provided herein, the actuator comprises a bridge structure with more than one fixed point. In some embodiments of aspects provided herein, the actuator is movable substantially parallel to a plane of the substrate. In some embodiments of aspects provided herein, the at least one nanoelectrode pair comprises a plurality of nanoelectrode pairs, and gaps between nanoelectrodes of the plurality of nanoelectrode pairs are adjustable by the same actuator. In some embodiments of aspects provided herein, the actuator is driven by thermal expansion. In some embodiments of aspects provided herein, the actuator comprises a bimetal deflection element. In some embodiments of aspects provided herein, the thermal expansion is driven by a heater element integrated into the substrate. In some embodiments of aspects provided herein, the thermal expansion is driven by a heater element external to the substrate.

Another aspect of the present disclosure provides a device for biopolymer sequencing, comprising: a substrate comprising at least one fluidic nanochannel; a plurality of electrode structures disposed on the substrate, each electrode structure comprising at least one nanoelectrode pair, each nanoelectrode pair having a region defining a gap between nanoelectrodes of the at least one nanoelectrode pair; an actuator that is integrated with the at least one nanoelectrode pair, which actuator adjusts a spacing of the gap between the nanoelectrodes of the at least one nanoelectrode pair; a data processor in electrical communication with the nanoelectrodes of the at least one nanoelectrode pair, wherein the data processor identifies a sequence of the biopolymer using electrical current across the gap.

In some embodiments of aspects provided herein, the substrate is silicon. In some embodiments of aspects provided herein, the actuator is a piezoelectric element incorporated into the substrate. In some embodiments of aspects provided herein, the actuator is a piezoelectric element external to the substrate. In some embodiments of aspects provided herein, the gap is oriented at an angle substantially non-perpendicular to the substrate plane. In some embodiments of aspects provided herein, the actuator comprises a cantilever structure. In some embodiments of aspects provided herein, the actuator comprises a bridge structure with more than one fixed point. In some embodiments of aspects provided herein, the actuator is movable substantially parallel to the plane of the substrate. In some embodiments of aspects provided herein, the at least one nanoelectrode pair comprises a plurality of nanoelectrode pairs, and gaps between nanoelectrodes of the plurality of nanoelectrode pairs are adjustable by the same actuator. In some embodiments of aspects provided herein, the actuator is driven by thermal expansion. In some embodiments of aspects provided herein, the actuator comprises a bimetal deflection element. In some embodiments of aspects provided herein, the thermal expansion is driven by a heater element integrated into the substrate. In some embodiments of aspects provided herein, the thermal expansion is driven by a heater external to the substrate. In some embodiments of aspects provided herein, the data processor is included in an external computing device. In some embodiments of aspects provided herein, the external computing device is a cloud computing device. In some embodiments of aspects provided herein, the electrical current is tunneling current.

Another aspect of the present disclosure provides a system for determining the sequence of a biopolymer, comprising: a substrate comprising at least one fluidic channel; a plurality of electrode structures disposed on or adjacent to the substrate, wherein each electrode structure of the plurality comprises at least one electrode pair separated by a gap, and wherein the at least one electrode pair intersects the at least one fluidic channel; and an actuator integrated with the at least one electrode pair, wherein the actuator controllably adjusts a spacing of the gap.

In some embodiments of aspects provided herein, the biopolymer is a nucleic acid molecule that is threaded through at least a portion of the gap. In some embodiments of aspects provided herein, the system further comprises a control system in electrical communication with the at least one electrode pair, which control system measures electrical current between the at least one electrode pair upon flow of the nucleic acid molecule through the gap. In some embodiments of aspects provided herein, the electrical current is tunneling current. In some embodiments of aspects provided herein, at least a subset of the plurality of electrode structures comprises a plurality of electrode pairs. In some embodiments of aspects provided herein, the plurality of electrode pairs are independently addressable. In some embodiments of aspects provided herein, the at least one of electrode pair is independently addressable.

Another aspect of the present disclosure provides a method for calibrating a plurality of electrodes for sequencing a nucleic acid molecule having monomers, comprising: providing a substrate with a nanochannel, wherein the nanochannel includes a pair of nanoelectrodes separated by a gap, wherein the gap has an adjustable spacing; flowing a plurality of reference calibration moieties in the nanochannel, wherein the reference calibration moieties correspond to at least some of the monomers of the nucleic acid molecule, and wherein the reference calibration moieties are non-nucleic acid moieties; using the nanoelectrodes to measure an electrical current through at least a subset of the plurality of reference calibration moieties; and based on measurement(s) of the electrical current, adjusting the spacing of the gap.

In some embodiments of aspects provided herein, the spacing is adjusted such that the electrical current measured with the nanoelectrodes corresponds to a predetermined electrical current profile. In some embodiments of aspects provided herein, the nanochannel includes a plurality of pairs of nanoelectrodes having gaps. In some embodiments of aspects provided herein, the method further comprises selecting pairs of nanoelectrodes, each of which is set to desirable gap spacing as determined by measurement of electrical current for the plurality of reference calibration moieties with the pairs of nanoelectrodes. In some embodiments of aspects provided herein, gaps that do not have a desirable nanoelectrode pair gap spacing are adjusted and the electrical current is measured upon flow of the plurality of reference calibration moieties through the nanochannel. In some embodiments of aspects provided herein, pairs of nanoelectrodes that do not have a desirable nanoelectrode pair gap spacing are flagged and data collection from the pairs of nanoelectrodes is stopped. In some embodiments of aspects provided herein, the method further comprises adjusting a spacing for each of the gaps based on measurements of electrical current with each of the pairs of nanoelectrodes upon flow of the reference calibration moieties through the nanochennel. In some embodiments of aspects provided herein, several subsets of the plurality of pairs of nanoelectrodes are formed of different metals, coatings or moieties associated thereto, and the measurement(s) of the electrical current yield a calibration setting(s) with different calibration values associated with each of the subsets of the plurality of pairs of nanoelectrodes. In some embodiments of aspects provided herein, the plurality of reference calibration moieties are synthetic or natural biopolymers containing a known sequence. In some embodiments of aspects provided herein, the plurality of reference calibration moieties have a symmetrical sequence. In some embodiments of aspects provided herein, the measurement(s) of the electrical current provides a calibration setting that is stored in non-volatile memory. In some embodiments of aspects provided herein, the non-volatile memory is on an instrument that (i) includes or is in proximity to the nanochannel, or (ii) is situated remotely with respect to the nanochannel. In some embodiments of aspects provided herein, the non-volatile memory is battery backed-up. In some embodiments of aspects provided herein, the calibration setting includes one or more calibration values that are retained for individual electrode pairs. In some embodiments of aspects provided herein, the reference calibration moieties are supplied as part of a kit. In some embodiments of aspects provided herein, the reference calibration moieties correspond to homopolymer sequences.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “Fig.” herein), of which:

FIG. 1 illustrates an overlapped adjustable nanogap with an overlay of an additional metal on the overlapping electrode.

FIG. 2 illustrates another overlapped adjustable nanogap with an overlay of an additional metal on the overhanging electrode where the gap is substantially planar.

FIG. 3 illustrates an inclined adjustable nanogap with an overlay of an additional metal on the upper electrode where both electrodes are cantilevered.

FIG. 4 illustrates an inclined adjustable nanogap with an underlay of an additional metal on the lower electrode where only the lower electrode is cantilevered.

FIG. 5 illustrates an inclined adjustable nanogap with an underlay of an additional metal on the lower electrode where both electrodes are at least partly cantilevered.

FIG. 6 illustrates another inclined adjustable nanogap with an underlay of an additional metal on the upper electrode where both electrodes are at least partly cantilevered.

FIG. 7 illustrates an inclined adjustable nanogap with a region with a temperature dependent material under the lower electrode and an integrated heater.

FIG. 8 illustrates an inclined adjustable nanogap with a piezo actuator integrated into the lower electrode.

FIG. 9 illustrates a top view of a horizontal actuator with two metals edge to edge in the cantilevered electrode.

FIG. 10 illustrates a side view of a horizontal actuator with two metals edge to edge in the cantilevered electrode.

FIG. 11 illustrates a top view of a horizontal actuator with a thermal expansion actuator moving the cantilevered electrode.

FIG. 12 illustrates a side view of a horizontal actuator with a thermal expansion actuator moving the cantilevered electrode.

FIG. 13A illustrates a side view of before electrode formation from vertical movement of a cantilevered electrode.

FIG. 13B illustrates a side view of a narrowed electrode during electrode formation from vertical movement of a cantilevered electrode.

FIG. 13C illustrates a side view of break after electrode formation from vertical movement of a cantilevered electrode with the gap adjusted to the desired spacing.

FIG. 14A illustrates a side view of several inclined adjustable nanogaps with an actuator.

FIG. 14B illustrates a top view of several inclined adjustable nanogaps with one actuator associated with each electrode pair.

FIG. 14C illustrates a top view of several inclined adjustable nanogaps with one actuator associated with multiple electrode pairs.

FIG. 15 schematically illustrates a computer system that is programmed or otherwise configured to implement devices, systems and methods of the present disclosure.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “gap,” as used herein, generally refers to a pore, channel or passage formed or otherwise provided in a material. The material may be a solid state material, such as a substrate. The gap may be disposed adjacent or in proximity to a sensing circuit or an electrode coupled to a sensing circuit. In some examples, a gap has a characteristic width or diameter on the order of 0.1 nanometers (nm) to about 1000 nm. A gap having a width on the order of nanometers may be referred to as a “nano-gap” (also “nanogap” herein). In some situations, a nano-gap has a width that is from about 0.1 nanometers (nm) to 50 nm, 0.5 nm to 30 nm, or 0.5 nm or 10 nm, 0.5 nm to 5 nm, or 0.5 nm to 2 nm, or no greater than 2 nm, 1 nm, 0.9 nm, 0.8 nm, 0.7 nm, 0.6 nm, or 0.5 nm. In some cases, a nano-gap has a width that is at least about 0.5 nm, 0.6 nm, 0.7 nm, 0.8 nm, 0.9 nm, 1 nm, 2 nm, 3 nm, 4 nm, or 5 nm. In some cases, the width of a nano-gap can be less than a diameter of a biomolecule or a subunit (e.g., monomer) of the biomolecule.

The term “electrode,” as used herein, generally refers to a material or part that can be used to measure electrical current. An electrode (or electrode part) can be used to measure electrical current to or from another electrode. In some situations, electrodes can be disposed in a channel (e.g., nanogap) and be used to measure the current across the channel. The current can be a tunneling current. Such a current can be detected upon the flow of a biomolecule (e.g., protein) through the nano-gap. In some cases, a sensing circuit coupled to electrodes provides an applied voltage across the electrodes to generate a current. As an alternative or in addition to, the electrodes can be used to measure and/or identify the electric conductance associated with a biomolecule (e.g., an amino acid subunit or monomer of a protein). In such a case, the tunneling current can be related to the electric conductance.

The term “biomolecule,” as used herein generally refers to any biological material that can be interrogated with an electrical current and/or potential across a nano-gap electrode. A biomolecule can be a nucleic acid molecule, protein, or carbohydrate. A biomolecule can include one or more subunits, such as nucleotides or amino acids.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double stranded.

The term “protein,” as used herein, generally refers to a biological molecule, or macromolecule, having one or more amino acid monomers, subunits or residues. A protein containing 50 or fewer amino acids, for example, may be referred to as a “peptide.” The amino acid monomers can be selected from any naturally occurring and/or synthesized amino acid monomer, such as, for example, 20, 21, or 22 naturally occurring amino acids. In some cases, 20 amino acids are encoded in the genetic code of a subject. Some proteins may include amino acids selected from about 500 naturally and non-naturally occurring amino acids. In some situations, a protein can include one or more amino acids selected from isoleucine, leucine, lysine, methionine, phenylalanine, threonine, tryptophan and valine, arginine, histidine, alanine, asparagine, aspartic acid, cysteine, glutamine, glutamic acid, glycine, proline, serin and tyrosine.

The term “layer,” as used herein, refers to a layer of atoms or molecules on a substrate. In some cases, a layer includes an epitaxial layer or a plurality of epitaxial layers. A layer may include a film or thin film. In some situations, a layer is a structural component of a device (e.g., light emitting diode) serving a predetermined device function, such as, for example, an active layer that is configured to generate (or emit) light. A layer generally has a thickness from about one monoatomic monolayer (ML) to tens of monolayers, hundreds of monolayers, thousands of monolayers, millions of monolayers, billions of monolayers, trillions of monolayers, or more. In an example, a layer is a multilayer structure having a thickness greater than one monoatomic monolayer. In addition, a layer may include multiple material layers (or sub-layers). In an example, a multiple quantum well active layer includes multiple well and barrier layers. A layer may include a plurality of sub-layers. For example, an active layer may include a barrier sub-layer and a well sub-layer.

The term “adjacent” or “adjacent to,” as used herein, includes ‘next to’, ‘adjoining’, ‘in contact with’, and ‘in proximity to’. In some instances, adjacent to components are separated from one another by one or more intervening layers. For example, the one or more intervening layers can have a thickness less than about 10 micrometers (“microns”), 1 micron, 500 nanometers (“nm”), 100 nm, 50 nm, 10 nm, 1 nm, or less. In an example, a first layer is adjacent to a second layer when the first layer is in direct contact with the second layer. In another example, a first layer is adjacent to a second layer when the first layer is separated from the second layer by a third layer.

The term “substrate,” as used herein, refers to any workpiece on which film or thin film formation is desired. A substrate includes, without limitation, silicon, germanium, silica, sapphire, zinc oxide, carbon (e.g., graphene), SiC, AlN, GaN, spinel, coated silicon, silicon on oxide, silicon carbide on oxide, glass, gallium nitride, indium nitride, titanium dioxide and aluminum nitride, a ceramic material (e.g., alumina, AlN), a metallic material (e.g., molybdenum, tungsten, copper, aluminum), and combinations (or alloys) thereof. A substrate can include a single layer or multiple layers.

### Devices for Detecting or Sensing Biomolecules

Nanogap tunneling electrodes have been used for measurement of DNA bases. When an appropriate gap size is selected, it is possible not just to detect the presence of DNA strands, but to determine the base sequence. But fabricating appropriate gap spacing for each nanogap is quite difficult, particularly when an array of electrodes is to be fabricated together; individual adjustment using an external XYZ mechanism is not possible due to size constraints. The tolerance which is needed is significantly less than a nanometer, and a tolerance of 0.1 nanometer (nm) or less may be desirable.

A system for biomolecule detection or sensing, including nucleic acid (e.g., DNA) sequencing, may be effectuated by a device with one or more fluidic nanochannels which incorporate nanoelectrode pairs that intersect the nanochannel combined with actuators which may serve as adjustors for one or more nanoelectrode pairs in order to adjust the nanoelectrode pairs to desired or optimal gap spacings. In some embodiments the nanogap tunneling electrodes may be fabricated on a substrate.

The substrate may be a chip of a semiconductor material such as: silicon, germanium, and the like; an isolating material such as a plastic material, glass, and the like; or a metal or an alloy of metals. If the substrate is made of a metal, the electrodes may be electrically isolated from the nanoelectrodes. In some embodiments the substrate may be polyamide.

In some embodiments the device may be fabricated in a MEMS facility. The device may include additional electronic circuitry, which may include transconductance amplifiers, analog to digital devices, computing devices, communications devices, memory devices, and any other hardware and or software components which may be useful for the collection, communication, storage and analysis of data which may be generated by the device.

The data from the device may be processed by a computer or data processor. In some embodiments the data may be processed by an external computing device. In some embodiments the data may be processed by a cloud computing device. In some embodiments GPU clusters may be used to process the data.

Space constraints for such a device are considerable, as it may be desirable to utilize a number of nanogap tunneling electrode pairs along a single nanochannel so as to read a single DNA strand several times in the process of sequencing said DNA strand. Further, a number of nanochannels may be utilized so as to detect measure and sequence multiple stands simultaneously in the different nanochannels.

The present disclosure provides several approaches of adjustment. One approach is to utilize temperature dependent adjustors in association with the nanogap pair, and the gap spacings may be adjusted utilizing changes in the temperature of the local electrode system, or by adjusting the temperature of a set of nanoelectrode pairs. In some embodiments, a piezo adjustor may be utilized, wherein a voltage impressed across a piezo crystal may be utilized to adjust one or more nanoelectrode pairs. Another approach is to utilize bimetallic cantilevers in association with the nanogap pair, wherein one or both electrodes are mounted on a cantilever, and the cantilevers may be adjusted utilizing changes in the temperature of the cantilever, wherein the difference in the thermal expansion coefficients results in a bending of the cantilever. Some materials with different thermal expansion coefficients may be utilized, which include gold with a coefficient of 14.3*10⁻⁶/K, aluminum with a coefficient of 23.1*10⁻⁶/K, silicon with a coefficient of 3.0*10⁻⁶/K, and tungsten with a coefficient of 4.3*10⁻⁶/K. In some embodiments the bimetal cantilever may be a bimetallic deflection mechanism.

For example, temperature induced deflection utilizing an lower layer of silicon nitride (temperature coefficient 3.2*10⁻⁶/K) and an overlaying layer of aluminum (temperature coefficient 23*10⁻⁶/K) and a length of 50 μm which experiences a temperature increase of 10 K will undergo a differential expansion between the of: (23−3.2)*10⁻⁶/K*5*10⁻⁵ m*10 K=9.9*10⁻⁹ m.

In some embodiments, the temperature of the structure may be raised utilizing an external source, such as an infrared (IR) source, which may be an IR laser or a near IR laser, or an external resistor, which may be coupled to one or more nanoelectrode pairs. Additional approaches for increasing the temperature of a structure include local resistors, which may allow temperature control over individual cantilevers, or groups of cantilevers. In some embodiments, the temperature of the structure may be raised by a heater integrated with the substrate. In some embodiments, a thermal expansion may be driven by in internal or external heater.

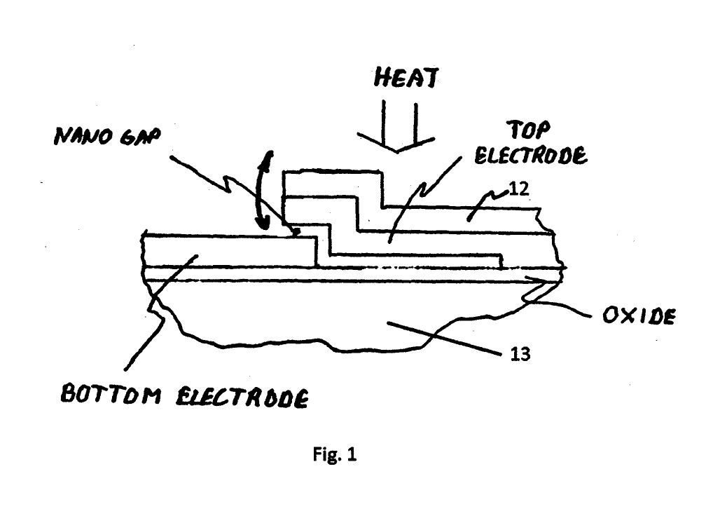

FIG. 1 schematically illustrates such a cantilevered nanoelectrode pair, wherein both the top and bottom electrode are fabricated of gold, although other metals may be used, while the overlaying (top) electrode has a layer of additional material 12, which may be a metal deposited on the gold top electrode. The overlaying metal may be fabricated of a material which has a larger expansion coefficient than that of gold, so that when the structure is heated, the cantilevered structure will bend down towards the bottom electrode, allowing adjustment of the nanoelectrode gap spacing. It is desirable in this embodiment that the initial gap spacing of the nanoelectrode pair be greater than desired so that the nanoelectrode gap spacing may be reduced to the appropriate spacing. As shown in FIG. 1, the electrodes may be fabricated on an optional layer of oxide, which may be silicon dioxide; alternatively the electrodes may be fabricated directly on the substrate 13, or may be fabricated on other structures, which may include additional layers which may be utilized for transistors, capacitors or other active portions of a semiconductor circuit, which may be planarized, potentially utilizing a dielectric material so as to prevent the gold or other metal from interfering with the proper operation of the semiconductor circuit.

While cantilevers may provide an embodiment which may allow for simpler fabrication, gap adjustors may be fabricated using electrodes supported at more than one point (bridge) or supported along a line such as a diaphragm.

The nanogap of FIG. 1 may be formed as part of the process which forms the cantilever associated with the top nanoelectrode, or may be made separately, either before or after removal of the layer which supported the top nanoelectrode, and is/was removed to form the cantilever. The layer which is removed to form the cantilever may be configured to overlay the bottom nanoelectrode, and the top electrode may be deposited on this layer such that the top nanoelectrode conforms to the change in elevation, allowing both similar interfaces to the underlying oxide or substrate 13, and allowing the top nanoelectrode to overlay the bottom electrode. The thickness of the layer between the top and bottom nanoelectrodes thus sets the initial spacing of the gap between the nanoelectrode pair.

In other embodiments, the gap between the nanoelectrodes may initially be a distance less than desired, and may then be increased by heating the structure. In this embodiment, it may be desirable that the layer of metal or other material have a coefficient of expansion which is less than that of the top nanoelectrode, such that when heated, the top nanoelectrode bends upward, increasing the gap spacing between the nanoelectrodes.

In some embodiments, the nanoelectrodes and or second materials 12 may have an additional dielectric layer formed, deposited or otherwise overlaid or underlying the nanoelectrodes such that the nanoelectrodes are not exposed to a fluid reagent except at the tips of the nanoelectrodes, thus preventing unwanted interactions between the nanoelectrodes and the fluid reagent. The tips may be configured so as to intersect the nanochannel, which may be a fluidic nanochannel, orthogonally or approximately orthogonally.

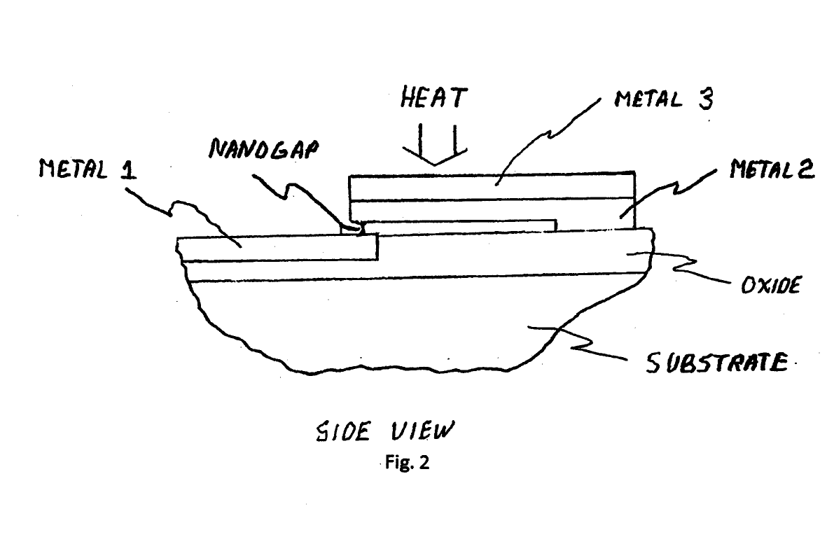

FIG. 2 schematically illustrates a similar structure to that of FIG. 1, but which utilizes three different materials, wherein the material of the two nanoelectrodes of the nanoelectrode pair may be of different metals (metal 1 and metal 2), and a third metal (metal 3) may be utilized to effect temperature dependent deflection of the cantilever. Metal 1 is depicted as being of the same height as an underlying oxide layer, which may have been grown to match the height of the previously deposited metal 1. A sacrificial layer may be deposited, followed by deposition of metal layer 2 and metal layer 3; the sacrificial layer may thence be removed, forming the nanogap and cantilever structure.

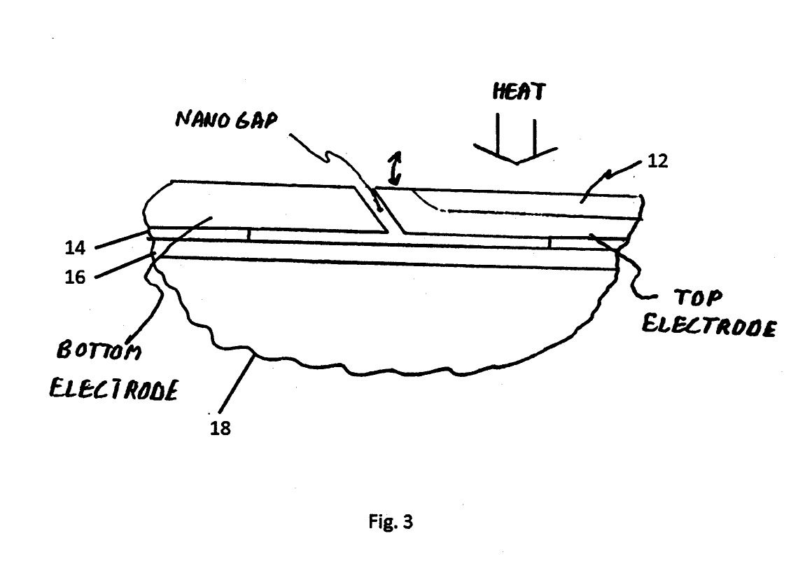

FIG. 3 schematically illustrates a structure that is similar to that shown in FIG. 1, but instead of having an overlay of a top nanoelectrode over a bottom nanoelectrode, the gap is formed using an angle. Such an angle may be formed using a reactive ion etch or other appropriate semiconductor fabrication methodology. Thus the two nanoelectrodes of the nanoelectrode pair may be initially fabricated using a single metalization step, instead of the two metalization steps as required by the structure of FIG. 1 to create the nanoelectrodes, and an additional metalization step to create the additional metal layer which results in temperature dependent deflection. FIG. 3 further illustrates both electrodes as being formed so as to create cantilevers, thus removing the need for alignment between any edge needed to form a cantilever associated with only one nanoelectrode, and the gap between the newly formed nanoelectrode pair. Similarly, the second material 12, which may be aluminum or other material which has a coefficient greater than that of the nanoelectrode for an embodiment wherein the gap initially is larger than desired. The temperature of the nanoelectrodes may be raised, bending the nanoelectrode depicted with the additional overlaying second material 12, and thus closing the gap between the nanoelectrodes. The overlaying second material 12 is depicted as not being extended to the gap between the nanoelectrodes; this may be done such that alignment with the gap between the nanoelectrodes is not required. In further embodiments, the second material may extend over part of both electrodes, insofar as there is sufficiently more material on one electrode than the other electrode as to allow differential bending which allows the gap between the nanoelectrodes to be set at the desired spacing. Any variability in the length of the cantilevers and or lengths of the overlaying second material 12 and or initial gap spacing between the nanoelectrodes may be compensated for by calibration of the temperature needed to close or open the nanoelectrode gap spacing to the desired spacing. In other embodiments similar to those previously described, the gap spacing may instead be narrower than desired, and an overlaying second material may be utilized which has a coefficient of expansion less than that of the electrode, such that when the structure is heated, the cantilever bends up, opening the gap spacing between the nanoelectrodes to the desired spacing.

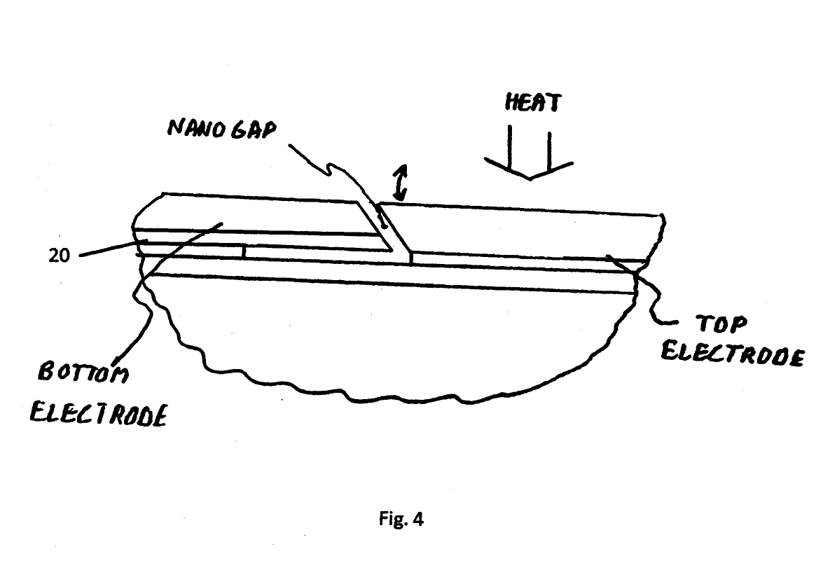

FIG. 4 schematically illustrates a structure similar to that of FIG. 3; an underlying material 20 is used under one electrode; although depicted as being aligned with the gap between the nanoelectrodes, the underlying material 20 may be applied such that it not be extended fully to the gap between the nanoelectrodes, or may be extended past the gap between the nanoelectrodes. Similarly, only one nanoelectrode is depicted as being cantilevered, requiring that removal of the underlying material is aligned with the location of the gap between the nanoelectrodes. In alternative embodiments, the second electrode may be partly undercut, forming a second cantilever, which may be of the same length as the depicted cantilever of the first nanoelectrode, or may be shorter. Any combination of cantilever length and or second material under or overlayment may be utilized in combination to create temperature controlled differential bending. A third or further additional materials may additionally be utilized, wherein the combined structure has a temperature dependent gap spacing between the nanoelectrodes.

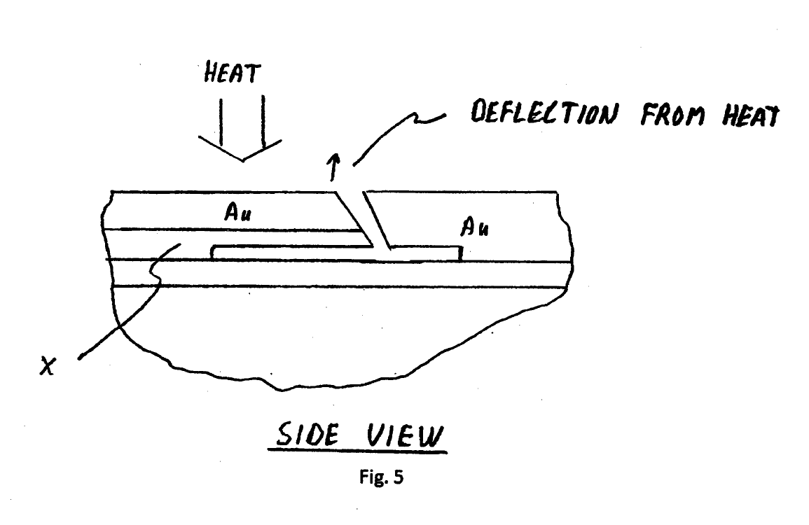

FIG. 5 schematically illustrates a structure that is similar to that shown in FIG. 4, wherein the underlying material, which may be of a material which may form a temperature dependent cantilever structure in combination with an overlaid electrode material, is deposited over a sacrificial layer (not shown) which is subsequently removed to form the cantilever. The material, which may be a metal different from that of the electrode material(s), may be planarized prior to deposition of the electrode material. The electrode material may thence be planarized prior to use a reactive ion etch or other methodology to cut the inclined nano gap. Alternatively, the gap may be cut prior to planarizing the electrodes. The gap is presumed in this case to be larger than desired.

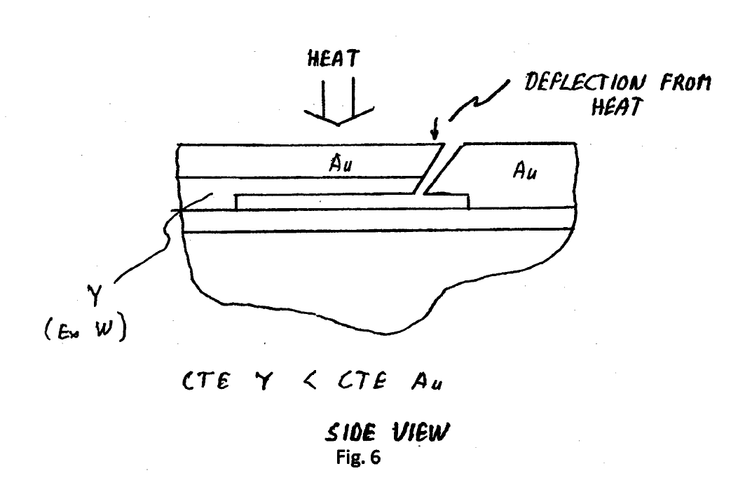

FIG. 6 schematically illustrates a structure that is similar to that shown in FIG. 5 with two differences: the direction of the angled cut which forms the nanogap is reversed, and the temperature coefficient of the underlying material relative to the electrode material is reversed, causing the cantilever structure to bend down with increasing temperature to close the gap, instead of bending with increasing temperature up to close the gap. In still further embodiments, wherein the gap which is cut is smaller than desired, the orientation of the gap or the temperature coefficient of the material other than the electrode relative to the temperature coefficient of the electrode material may be reversed such that the cantilevered electrode may bend up or down with increasing temperature to open the gap between the nanoelectrodes to the desired gap spacing.

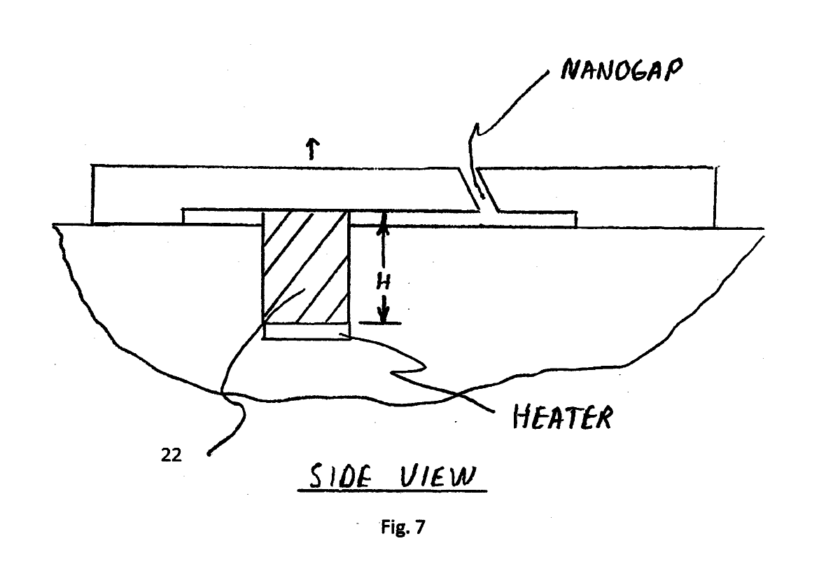

In other embodiments, instead utilizing a difference in expansion coefficients between the electrode and an another material to bend the electrode in an arc as a result of the materials being bonded together and not being constrained such as when an extended cantilever is fabricated, a material may be used which is constrained between a comparatively inflexible substrate and an electrode structure. FIG. 7 schematically illustrates such a device, wherein a heating element is depicted under a material with a temperature dependent expansion coefficient. The temperature dependent material 22 is depicted in conjunction with a cantilevered electrode, and is shown as being closer to the portion of the electrode which is in contact with the substrate relative to the electrode tip. If the temperature dependent material is bound to the electrode, the tip will be lifted by the amount the temperature dependent material 22 increases in its vertical dimension relative to the electrode and the substrate, as shown in this depiction, the relative temperature dependence is dominated by the substrate material rather than the electrode material. In this embodiment, the electrode will deform in the region between the temperature dependent material 22 and the attachment of the electrode to the substrate. In other embodiments, wherein the temperature dependent material is not bound to the electrode, the movement of the electrode tip will be magnified by the lever action of the electrode relative to the distance between the contact point between the temperature dependent material 22 and the electrode and the electrode tip, and the contact point between the temperature dependent material 22 and the point at which the electrode is bound to the substrate. Thus the distance the nanoelectrode tip moves relative to a temperature change may be changed at the time of fabrication by changing the lever configuration, moving either the location of the temperature dependent material, or the location of the inclined cut which forms the gap between the nanoelectrodes. The length of the cantilevered portion of a nanoelectrode may also be increased or decreased.

In some embodiments the system which may include a cantilever or other temperature dependent element, the cantilever or other temperature dependent element may be heated using integrated resistive heating, external resistive heating, electromagnetic radiation such as laser light, or other types of heating. In some embodiments temperature can be measured locally by thermistors, resistive thermal devices and the like. In other embodiments the desired position can be inferred from tunneling current measurements. The system may be fabricated such that a gap only exists at temperatures above room temperature.

In some embodiments a sharp pair of tips may be formed by stretching to breakage a small trace, which may have been formed using lithographic techniques, and may be configured to be suspended over a trench, thus initially forming the nanoelectrode tips. This can be done in a single step or by multiple stress cycles.

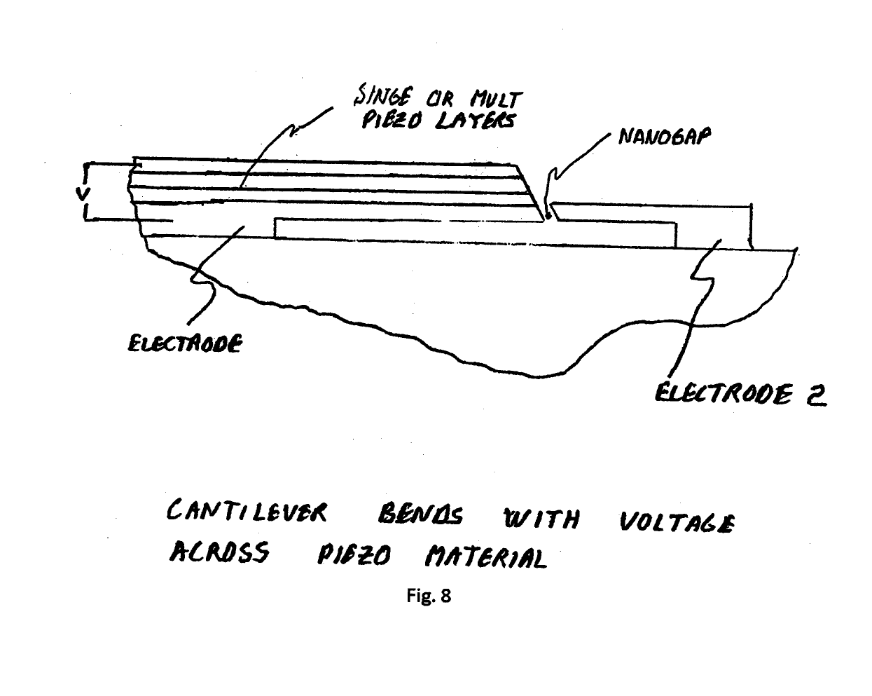

In some embodiments, a piezo material may be utilized instead of a material with a different expansion coefficient than the electrode material. FIG. 8 schematically illustrates a side view of an integrated piezo gap adjuster. In some embodiments the piezo actuator may be integrated into the electrode. In some embodiments the nanogap nanoelectrode material is used to set the voltage across one element of the piezo stack, while a separate electrode may be used for the opposite side of the piezo material so as to set a differential voltage between the voltage of the nanoelectrode and the additional electrode. In other embodiments two additional electrodes separate from the nanoelectrodes may be used is used to set the voltage. The piezo actuator may be comprised of one or more piezoelectric materials (“piezo”), and additional electrodes may be formed between different piezo elements of the stack. In some embodiments a top electrode may be of a different thickness than a bottom electrode of a piezo element. The piezo stack may be oriented such that the direction of expansion or contraction of the piezo material is parallel to the cantilever arm. As voltage is applied the piezo stack may expand causing the electrode support to bend. This bending may provide adjustment of the electrode gap, and may be configured to bend such the gap may be opened or closed.

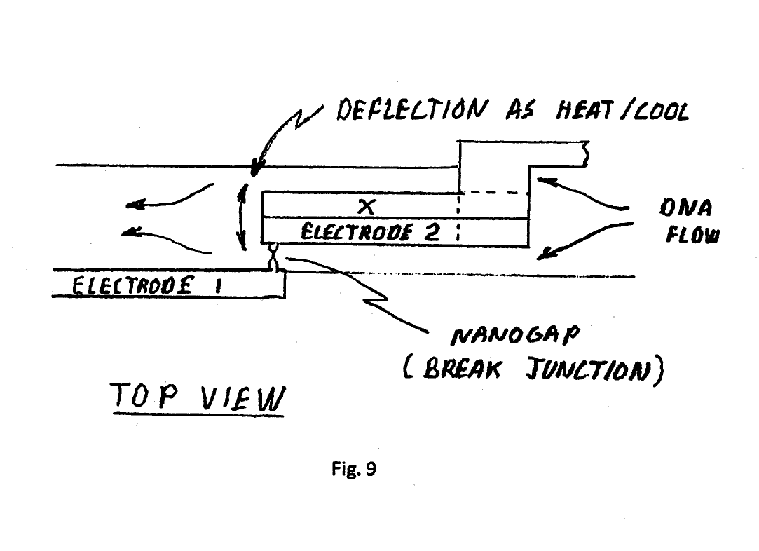

FIG. 9 schematically illustrates a top view of a horizontal gap adjuster. In this embodiment two or more metals with different coefficients of thermal expansion may be used. For example electrode 1 and electrode 2 may be fabricated of gold with a CTE of 14.3*10̂6/K and a second material of electrode 2 may be tungsten (CTE of 4.3*10̂6/K); an increase in the temperature will cause the cantilever of electrode 2 to bend away from electrode 1 causing the small connection between the electrodes to break, or a gap spacing to be increased. In other embodiments the materials may be arranged with different expansion coefficients such that an increase in temperature may cause the gap between the nanoelectrodes to be decreased.

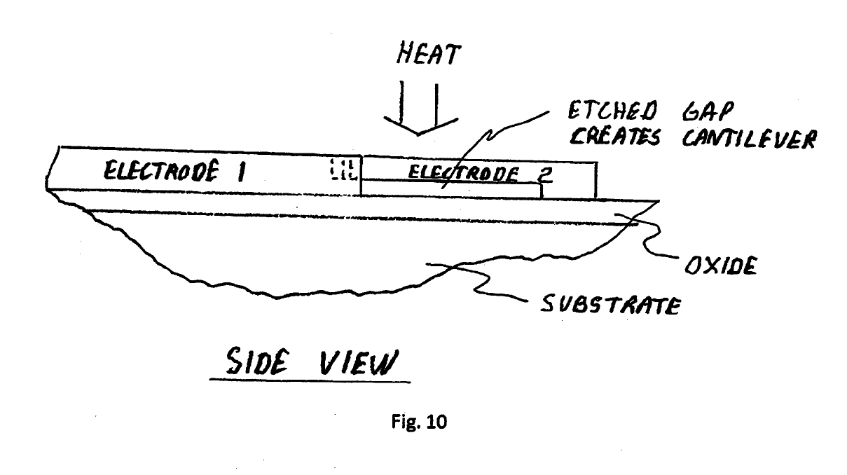

FIG. 10 schematically illustrates a side view of a horizontal bimetal nanoelectrode gap adjuster. The gap depicted below the nanoelectrodes may be formed by etching a sacrificial layer (not shown) facilitating motion of the cantilever. An optional layer of oxide is shown between the nanoelectrodes and the underlying substrate,

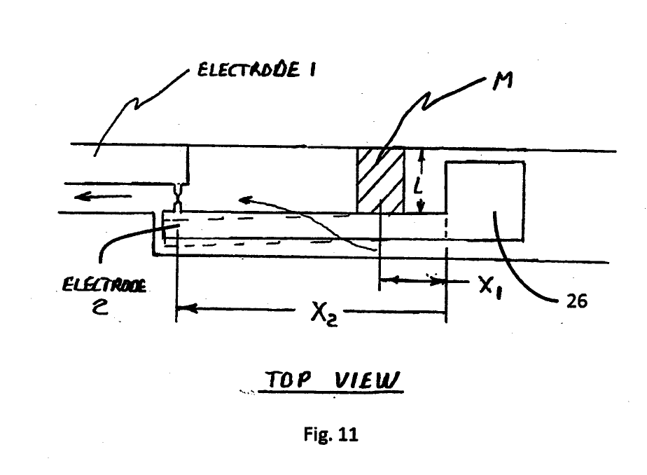

FIG. 11 schematically illustrates a top view of an embodiment of a gap adjustment system where motion may be in the horizontal plane. Nanoelectrode 2 is shown as having a cantilever section. A thermal expansion actuator M is shown positioned at a length X1 from the electrode base 26. This actuator may be comprised of a material with a coefficient of thermal expansion different from the substrate. In some embodiments the material may be a metal compatible with semiconductor processes such as aluminum or copper. In other embodiments the material may be an organic material such as paraffin, or plastics such as PMMA, or a resist such as SUB. The expansion/contraction of the thermal expansion actuator M may be amplified at least in part due to the lever arm ratio: X2/X1.

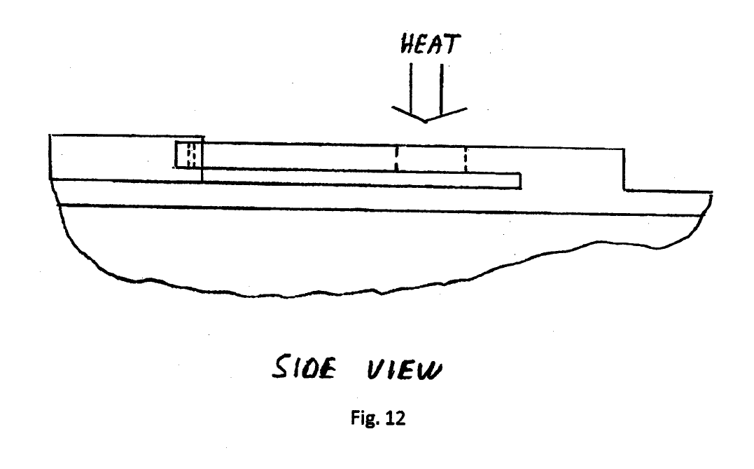

FIG. 12 schematically illustrates a side view of the horizontal gap adjuster depicted in FIG. 11. The gap below the nanoelectrodes may be formed by etching a sacrificial layer, thereby facilitating motion of the cantilever. In some embodiments the sacrificial layer may extend underneath the thermal actuator material, as well as the nanoelectrode.

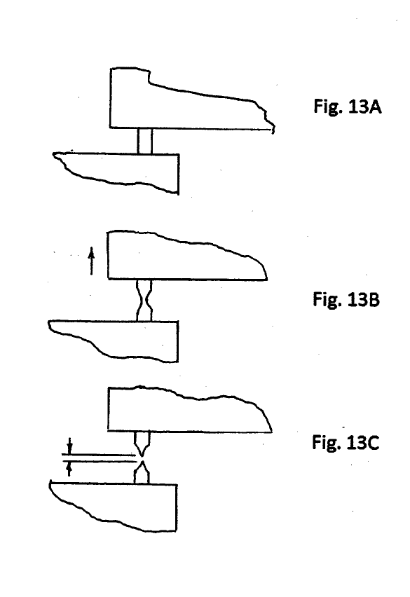

FIG. 13A-C schematically illustrates a method of fabrication of electrode tips in a vertical adjustment system for detection of tunneling currents using a nanoelectrode pair gap. In FIG. 13A, a via is formed across a sacrificial layer utilizing standard lithographic methods. In FIG. 13B the electrodes have been pulled apart by one of the gap adjustment mechanisms described hereinabove, causing the metal of the via to stretch, and the thickness of the metal of the via to neck down. In FIG. 13C the electrodes have been separated and the gap initially adjusted.

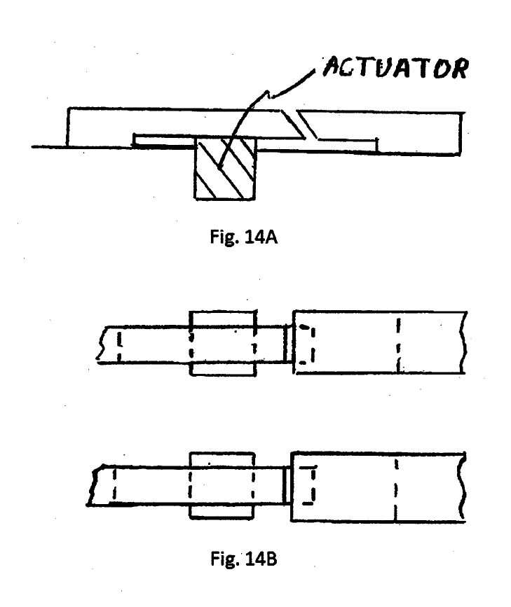

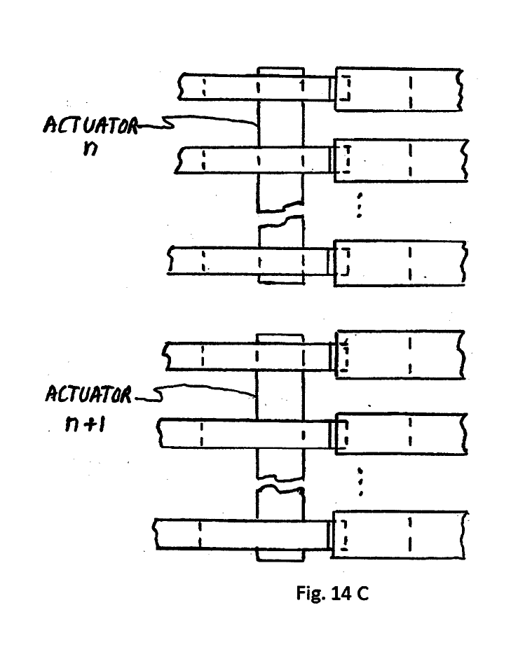

FIG. 14A schematically illustrates a side view of a nanoelectrode gap adjuster. The actuator may be a piezo device, a thermal expansion device or other actuator. FIG. 14B shows an embodiment wherein one actuator may be used for each several nanoelectrode pairs. FIG. 14C shows an embodiment where multiple nanoelectrode pair gaps may be adjusted by a single actuator, and multiple actuators may adjust multiple sets of nanoelectrode pair gaps. In an embodiment wherein a set of multiple nanoelectrode pair gaps are adjusted by a single adjuster, the actuator for the adjustor may adjust the set of multiple nanoelectrode gaps such that the average gap of the set of multiple nanoelectrode pair gaps is set to an ideal spacing. In other embodiments the set of multiple nanoelectrode pair gaps can be set so one or more of the set of multiple nanoelectrode pair gaps are set to an ideal spacing. In some embodiments test runs or controls may be used to determine which nanogaps are preferred, potentially as a result of having a set of the set of multiple nanoelectrode pair gaps having a similar nanoelectrode pair gap spacing, and data may be collected from a preferred subset of the set of multiple nanoelectrode pair gaps

### Calibrating Nanogap Electrodes

The present disclosure provides methods for calibrating nanoelectrodes, such as adjustable nanoelectrodes. Nanoelectrode pairs can be calibrated using calibration standard moieties. The calibration standard moieties may include a nucleic acid molecule (e.g., DNA or RNA) or a plurality of nucleic acid molecules, and may utilize natural or synthetic bases, which may include labels or other modifications to the nucleic acid polymer. This may be particularly desirable when the targets to be detected are DNA and or RNA or other similar natural or synthetic nucleic acids. Said calibration of the nanoelectrode pairs may be useful in optimizing the response of the nanoelectrode pairs for use in DNA sequencing or sequencing other biopolymers, or for identifying and quantifying the number of biopolymers or other desired targets in a solution. The tunneling current may pass through a calibration standard moieties and through a nanoelectrode pair which is physically directly associated thereby, which thereby measures the calibration standard. The calibration standard moiety may be physically between the nanoelectrodes of a nanoelectrode pair to be considered to be directly physically associated thereby.

Calibration standard moieties may comprise homopolymer nucleic acids. For example when utilizing a system comprising sets of nanoelectrode pairs for sequencing nucleic acids, it may be desirable to utilize calibration standards which comprise poly A, poly T, poly C, and poly G DNA, or poly A, poly U, poly G and poly C RNA. The calibration standard moieties may be introduced separately, so that the system can introduce, for example, only poly A oligonucleotides (“oligos”), and the nanoelectrode gaps may be set to a desirable spacing for the single homopolymer species. Thus, one or more additional homopolymer oligo species may be introduced as additional calibration standard moieties. Calibration standard moieties may be introduced by pressure driven fluidic flow into nanochannel(s) with which the nanoelectrode pair(s) are associated whereby the calibration standard moieties may interact directly with the nanoelectrode pairs such that tunneling current measurements may be taken of the calibration standard moieties by the nanoelectrode pairs, wherein the nanoelectrode pairs may be in fluidic contact with the solution within the nanochannel. Alternatively, calibration standard moieties may be introduced to the nanochannel(s) utilizing electrical forces, such as electokinetic and or electroendosmotic (or electroosmotic) forces. A number of nanoelectrode pairs may be configured so as to be in fluidic contact with each nanochannel.

The system may measure and adjust the nanoelectrode gap spacings so as to optimize the gap spacings. It may be desirable to have the gap spacing initially set to a spacing which is larger than a desired spacing, and thence reduce the gap spacing until a desired tunneling current level is measured when a homopolymer nucleic acid is utilized as a calibration standard. In some embodiments, a calibration standard moiety is used which produces a reliable signal when the gap spacing is set to the largest spacing which may produce a reliable signal, while other potential calibration standards, for example, other homopolymer nucleic acids, may produce a significantly smaller current with the same nanoelectrode pair gap spacing. The gap may then be adjusted so as to provide appropriate signal levels (e.g., signal to noise ratio, such as at least 5:1, 10:1, 100:1 or 1000:1) for other calibration standards, which may be homopolymer nucleic acid polymers that give the smallest tunneling current levels when the gap is at a desired position to optimally measure all four nucleic acids.

In other embodiments, a combination of homopolymer oligos are used at the same time. In such a case, the gap may be set as a result of monitoring tunneling currents which result from measuring two or more homopolymer nucleic acid calibration standards which may be provided in a single solution to the nanoelectrode pairs at the same time. The system may measure a number of different molecules before making adjustments so as to insure statistically or absolutely that an appropriate number of each type of calibration standard nucleic acid homopolymers have been measured.

In some embodiments, synthetic oligos comprising a known sequence, which may comprise homopolymer runs (i.e., sequences of bases) of various sorts, for example regions of poly G and poly U RNA, which may provide maximum and minimum signals for RNA molecules, may be utilized. Other combinations of homopolymer nucleic acid runs may be utilized.

In some embodiments, synthetic or natural oligos comprise a known sequence which may comprise combinations of homopolymer runs and sequences which do not comprise homopolymer runs. In some embodiments, synthetic or natural oligos comprising an unknown sequence may be utilized, wherein the nanoelectrode pair gap spacings may be set utilizing measurements of the unknown sequence data measurements. In some embodiments, synthetic or natural oligos comprising a symmetrical sequence may be utilized so that the signal is not dependent on orientation.

The supplied calibration standard moieties may be part of a calibration kit. The moieties utilized as calibration standard moieties may be an unknown sample, wherein data measurements acquired prior to achieving a desired level of calibration of the nanoelectrode gap spacings may be discarded. The moieties utilized as calibration standard moieties may be added to an unknown sample, wherein data measurements acquired prior to achieving a desired level of calibration of the nanoelectrode gap spacings may be discarded.

Other types of calibration standards moieties may be utilized, which may be different types of polymers, such as polypeptides, particularly when the target sample may be a polypeptide. Other types of polymers may be utilized as calibration standard moieties, corresponding to other different types of similar or identical target molecules, such as carbohydrate polymers, lipid polymers, or other biological polymers, or synthetic polymers. Monomers or other more complex molecules may be utilized as calibration standard moieties corresponding to different types of similar or identical target molecules. The calibration standard moieties may include repeats or homopolymers of at least some of the target biopolymers, or may be directly related in size and or tunneling current for a particular nanoelectrode gap spacing to desired monomers of a target biopolymer.

Calibration standard moieties that include molecules of different types than those that are to be measured may be utilized. For example, a target molecule may be a polypeptide, while the nanoelectrode gap spacing may be set (or calibrated) using calibration standard moieties comprising DNA homopolymers. The gap spacing may be set to a current level which corresponds directly to a desired gap spacing for the desired target molecule, while the nanoelectrode gap spacing may provide nonoptimal current levels for the calibration standard moieties.

In some embodiments, nanoelectrode pair gap spacings may be set utilizing calibration standard moieties, wherein the nanoelectrode pair gap spacing may thence be adjusted to a different nanoelectrode pair gap spacing. The different nanoelectrode pair gap spacing may be adjusted by a fixed previously determined amount, or may be adjusted based on measurements of two or more different types of measurements, which may be of optimal settings for different types of calibration standard moieties, wherein the difference in temperature, voltage or other applied adjustment parameter needed for optimal nanoelectrode pair gap spacings may be utilized to determine an appropriate change in temperature voltage or other applied adjustment parameter in order to adjust the nanoelectrode pair gap spacing to the desired gap spacing for the intended target molecule(s).

In some embodiments, one measurement may be utilized which may be of current levels and nanoelectrode gap spacings, wherein there is no calibration standard moiety supplied to the nanoelectrode pair gap, but instead current measurements are made of current directly between the nanoelectrodes. This may be utilized to directly set nanoelectrode pair gap spacing, or a desired nanoelectrode gap spacing may be determined by combining a measurement taken directly between the nanoelectrodes with measurements taken using calibration standard moieties.

In some embodiments, wherein nanoelectrode pair gap spacings may be set using measurements of other moieties or direct measurements between nanoelectrode pairs, changes in setting to control parameters may be determined by direct linear interpolation from known actual gap spacings, or from known relative control parameter changes, taken, for example from a table. Other methods may be utilized, for example calculating the desired changes using polynomial, logarithmic, exponential equations, combinations thereof, or any other appropriate mathematical representation of the nanoelectrode pair gap spacing movement as a function of changes in the control parameters, which could be an output which is a pulse width modulation, a digital to analog converter, a setting of an electrically adjustable potentiometer, a setting in a memory of a control circuit, or any other appropriate method for controlling a nanoelectrode pair gap spacing.

In some embodiments, calibrating may include a process of introducing a calibration standard(s), measuring said calibration standard(s), and adjusting a gap spacing(s) as appropriate to effectuate a desired gap spacing(s). In further embodiments, calibrating may include repeated measuring of said calibration standard(s) after said adjusting of gap spacing(s), and readjusting to effectuate a desired gap spacing(s).

In some embodiments, calibrations of nanoelectrode pair gap spacings may be performed in a factory, and may be made available at the point of use of the nanoelectrode pair spacing by storing calibration values in a non-transitory computer readable medium, such as memory, which may be flash memory or other nonvolatile memory directly physically associated with the nanoelectrode pairs, such as by fabricating nonvolatile memory directly on the same substrate as the nanoelectrode pairs, or by fabricating nonvolatile memory on a substrate which may thence be associated with the nanoelectrode pairs within a multi-chip module or hybrid circuit, or as part of printed circuit assembly. In some embodiments, nonvolatile memory may be directly physically associated with the nanoelectrode pairs and a battery.

In some embodiments, calibration values of nanoelectrode gap spacings may be stored in memory which is not directly physically associated with the nanoelectrode pairs, such as supplying a compact disc or DVD, supplying a memory stick, a hard drive, or any other appropriate data storage device. A device with a set of nanoelectrodes may be associated with the external data storage device utilizing barcoding, which may be optical, or may be made available utilizing blue tooth, Wi-Fi, or other wireless connectivity, or utilizing USB, Ethernet, CAN or other appropriate hardware connectivity.

In yet some embodiments, calibration values may be accessible from a central database, decentralized database, or cloud database, and may be associated with a device with a set of nanoelectrodes utilizing a barcode or other identification. Calibration values may be accessible over a network.

In some embodiments, calibration may be performed at the time the device with nanoelectrode pairs is utilized by an end user. Calibration values may be determined as described herein, and may be stored in an associated control system (see below), or storage memory associated with said associated memory system.

In some embodiments, nanoelectrode pair gaps may be formed as the device is utilized by an end user, wherein a nanoelectrode may be stretched and broken after installation of the nanoelectrode structure device by the end user, thus preventing any potential damage as a result of removal of gap spacing controls such as elevated temperatures or piezo voltages during shipment. In further embodiments, a battery, which may be a battery which is also utilized to maintain values in a volatile memory, may be utilized to maintain a piezo voltage or elevated temperature during shipment so as to prevent damage to nanoelectrode pairs as a result of removal of said elevated temperatures or piezo voltages, the removal of which may cause nanoelectrode tips to be deformed against each other, despoiling the tip structures created by pulling and breaking the nanoelectrode structures.

In some embodiments, adjustors may be fabricated such that there is one adjustor for each nanoelectrode pair, and thus each nanoelectrode pair gap spacing may be adjusted individually. In such embodiments, particularly for embodiments which utilize temperature dependent adjustors, there may be thermal crosstalk between different nanoelectrode pairs, and particularly between adjacent nanoelectrode pairs. Thus in some embodiments, it may be desirable to design the device so as to minimize thermal conduction between devices, and may be desirable to adjust the nanogaps together, so that thermal crosstalk may be compensated for as part of the calibration procedure. In some embodiments, it may not be possible to adjust all of the devices to an optimal or even to a desirable setting, and thus data from a device which cannot be appropriately set may be ignored.

In some embodiments, sets of nanoelectrodes may be selected as a result of the measurements of tunneling currents taken while calibration standard moieties are disposed in the gap spacing between the nanoelectrodes of a nanoelectrode pair. Said selection may result in discarding data from nonselected nanoelectrode pairs, or marking data associated with said nonselected nanoelectrode pairs as being from a nanoelectrode pair with a bap spacing which results in nonoptimal or less than desired tunneling current. Sets of nanoelectrodes with less desirable calibration values may be deselected and no longer used for data collection. The measurement of the tunneling currents associated with the nanoelectrode pairs may be stored so as to permit subsequent analysis algorithms to determine which sets of nanoelectrode data to utilize and or how to weight said sets of nanoelectrode data.

In some embodiments, a single adjustor may be utilized for several nanoelectrode pairs, fabrication variations in nanoelectrode gap spacings or nanoelectrode gap spacing response to adjustment by the associated nanoelectrode adjustor between nanoelectrode pairs may be sufficient as to prevent a single setting of the common adjustor to allow to adjustment of all of the devices to an optimal or even to a desirable setting, and thus it may desirable to set the adjustor to a position which allows the greatest number of devices to have an optimal nanoelectrode pair gap spacing, or it may be desirable to set the adjustor to a position which allows the greatest number of devices to have a desirable nanoelectrode pair gap spacing. Nanoelectrodes pair gap spacings which are not able to be set to an optimal or desired spacing may have resulting data from said nanoelectrode pairs discarded, or otherwise marked as being potentially questionable.

In some embodiments, it may be desirable to set different nanoelectrode pairs within a single nanochannel with different nanoelectrode pair gap spacings, such that some of the nanoelectrode pair gap spacings may be tuned to optimal or desirable for different target moieties; for example, some nanoelectrode pair gap spacings may be optimized for uridine nucleobases, while other nanoelectrode pair gap spacings may be optimized for guanosine nucleobases. Setting nanoelectrode pair gap spacings to be optimal for one nucleobase may allow better measurement data to be obtained for that base, or to better distinguish that nucleobase from another nucleobase, but may reduce the ability to differentiate between other pairs of nucleobases, thus having several different nanoelectrode pair spacings may be desirable. Similarly, if several different types of molecules are being measured simultaneously, such as for example, nucleic acids and polypeptides, it may be desirable to have different nanoelectrodes pair gap spacings set with quite different distances.

In some embodiments, different nanoelectrode pairs may be modified so as to have different metals or other surface coatings, such that the different nanoelectrode pairs may interact differently with different bases or other different target moieties. It may be desirable to set or calibrate the different nanoelectrode pairs to different physical gap spacings so as to optimize the response for the intended target moiety.

In some embodiments, different nanoelectrode pairs may be modified so as to have different interaction molecules bound thereto, such that the different nanoelectrode pairs with different interaction molecules bound thereto may interact differently with different bases or other different target moieties. It may be desirable to set or calibrate the different nanoelectrode pairs to different physical gap spacings so as to optimize the response for the intended target moiety. In some embodiments this may result from binding of the different interaction molecules to different locations upon the nanoelectrode pairs; in other embodiments this may result from different optimal physical nanoelectrode pair gap spacings as a result of the different physical size of different species of interaction molecules.

### Computer Systems

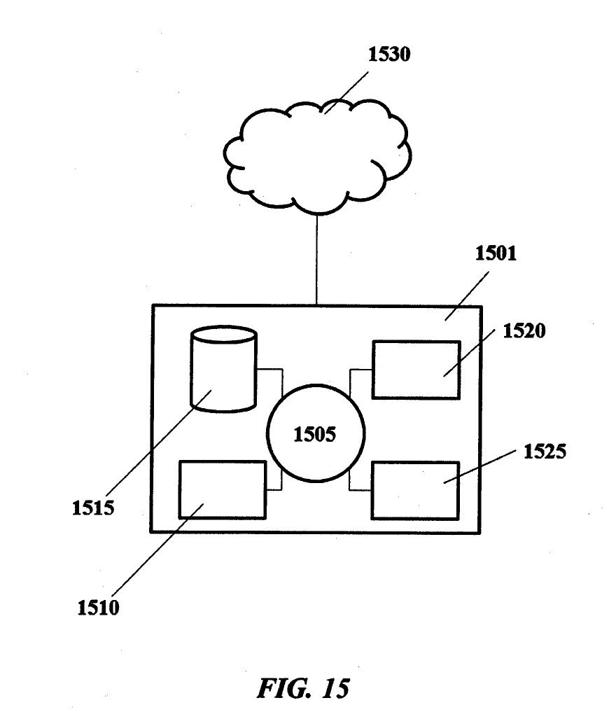

The present disclosure provides computer control systems that are programmed or otherwise configured to implement methods provided herein, such as calibrating sensors of the present disclosure. FIG. 15 shows a computer system 1501 that includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet. The computer system 1501 can communicate with one or more remote computer systems through the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

The computer system 1501 can be programmed or otherwise configured to regulate one or more processing parameters, such as the substrate temperature, precursor flow rates, growth rate, carrier gas flow rate and reaction chamber pressure. The computer system 1501 can be in communication with valves between the storage vessels and a reaction chamber, which can aid in terminating (or regulating) the flow of a precursor to the reaction chamber.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Methods and systems of the present disclosure can be implemented by way of one or more algorithms. An algorithm can be implemented by way of software upon execution by the central processing unit 1505.

Devices, systems and methods of the present disclosure may be combined with and/or modified by other devices, systems, or methods, such as those described in, for example, JP 2013-36865A, US 2010/0025249, US 2012/0193237, US 2012/0322055, US 2013/0001082, US 2014/0300339, JP 2011-163934A, JP 2005-257687A, JP 2011-163934A and JP 2008-32529A, each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A device for determining a sequence of a biopolymer, comprising:
a substrate comprising at least one fluidic nanochannel;
a plurality of electrode structures disposed adjacent to the substrate, each electrode structure of the plurality comprising at least one nanoelectrode pair, wherein each nanoelectrode pair comprises a region defining a gap between nanoelectrodes of the at least one nanoelectrode pair, and wherein the at least one nanoelectrode pair intersects the at least one fluidic nanochannel; and
an actuator that is integrated with the at least one nanoelectrode pair, which actuator adjusts a spacing of the gap between the nanoelectrodes of the at least one nanoelectrode pair.

2. The device of claim 1, wherein the substrate is silicon.

3. The device of claim 1, wherein the actuator is a piezoelectric element incorporated into the substrate.

4. The device of claim 1, wherein the actuator is a piezoelectric element external to the substrate.

5. The device of claim 1, wherein the gap is oriented at an angle substantially non-perpendicular to the substrate plane.

6. The device of claim 1, wherein the actuator comprises a cantilever structure.

7. The device of claim 1, wherein the actuator comprises a bridge structure with more than one fixed point.

8. The device of claim 1, wherein the actuator is movable substantially parallel to a plane of the substrate.

9. The device of claim 1, wherein the at least one nanoelectrode pair comprises a plurality of nanoelectrode pairs, and wherein gaps between nanoelectrodes of the plurality of nanoelectrode pairs are adjustable by the same actuator.

10. The device of claim 1, wherein the actuator is driven by thermal expansion.

11. The device of claim 10, wherein the actuator comprises a bimetal deflection element.

12. The device of claim 10, wherein the thermal expansion is driven by a heater element integrated into the substrate.

13. The device of claim 10, wherein the thermal expansion is driven by a heater element external to the substrate.

14. A device for biopolymer sequencing, comprising:
a substrate comprising at least one fluidic nanochannel;
a plurality of electrode structures disposed on the substrate, each electrode structure comprising at least one nanoelectrode pair, each nanoelectrode pair having a region defining a gap between nanoelectrodes of the at least one nanoelectrode pair;
an actuator that is integrated with the at least one nanoelectrode pair, which actuator adjusts a spacing of the gap between the nanoelectrodes of the at least one nanoelectrode pair;
a data processor in electrical communication with the nanoelectrodes of the at least one nanoelectrode pair, wherein the data processor identifies a sequence of the biopolymer using electrical current across the gap.

15.-26. (canceled)

27. The device of claim 14, wherein the data processor is included in an external computing device.

28. The device of claim 27, wherein the external computing device is a cloud computing device.

29. The device of claim 14, wherein the electrical current is tunneling current.

30. A system for determining the sequence of a biopolymer, comprising:
a substrate comprising at least one fluidic channel;
a plurality of electrode structures disposed on or adjacent to the substrate, wherein each electrode structure of the plurality comprises at least one electrode pair separated by a gap, and wherein the at least one electrode pair intersects the at least one fluidic channel; and
an actuator integrated with the at least one electrode pair, wherein the actuator controllably adjusts a spacing of the gap.

31.-52. (canceled)
